(19) United States
(12) Patent Application Publication
(10) Pub. No.: US 2022/0167594 A1
(43) Pub. Date: Jun. 2, 2022

(54) METHOD AND CONTROL ARRANGEMENT FOR DETECTING A HEALTH CONDITION OF AN ANIMAL

(71) Applicant: DELAVAL HOLDING AB, Tumba (SE)

(72) Inventor: Bohao LIAO, Tumba (SE)

(21) Appl. No.: 17/601,609

(22) PCT Filed: Mar. 23, 2020
(86) PCT No.: PCT/SE2020/050301

§ 371 Date: Oct. 5, 2021

(30) Foreign Application Priority Data
Apr. 5, 2019 (SE) 1950426-5

(51) Int. Cl. A01K 29/00; G06T 7/40; G06T 7/00; G06T 7/30; G01J 5/48; G01J 5/00; G01J 5/08

(52) U.S. Cl. CPC A01K 29/005; G06T 7/40; G06T 7/0012; G06T 7/30; G01J 5/48; A61B 5/015; G01J 5/0859; G06T 2207/10024; G06T 2207/30004; G01J 2005/0077; G01J 5/0025

(57) ABSTRACT

A method, controller, computer program, and computer program product are provided for determining a health condition of an animal by capturing a thermographic image of at least one portion of the animal and capturing a visible light image of the at least one portion of the animal, the visible light image corresponding to the thermographic image. The method further includes determining at least one surface property of the at least one portion of the animal based on the visible light image, adjusting the thermographic image to compensate for impact of the determined at least one surface property, and determining the health condition of the animal based on the adjusted thermographic image.

### TECHNICAL FIELD

The present disclosure generally relates to the field of farming and more specifically to methods and arrangements for determining a health condition of an animal.

### BACKGROUND

Like humans, livestock (e.g. cows, swine, sheep, etc.) are exposed to and experience a variety of disease, injury, illness, and other health conditions. It is generally desirable to treat the animals immediately upon learning of the onset of an illness or other health condition. Particularly for those whose livelihood depends on the survival of the animals they care for—e.g., farmers, ranchers, breeders, etc.—the health of animals under their care is of utmost concern. Breakouts of disease (e.g. infection, mastitis, influenza) can wipe out entire herds and/or otherwise adversely affect production of e.g. milk. For example, mastitis may have significant negative impact on milk productivity and quality with diagnosis of clinical mastitis often prompting isolation of animals from a herd and even emergency slaughter.

Thermal imaging is a good way to monitor an animal and has been used for detecting for example mastitis. Thermal imaging is non-invasive and has no significant running cost. In one procedure, a thermal camera measures surface temperature of an animal or specific part of animal. When an animal has a disease, the surface temperature may increase. One example of a heat camera system is shown in document WO2014/083433 A2.

However, thermal radiance varies with surface properties of animals, such as skin colour and hairiness, and different animals may have different skin/hair, which may consequently affect the thermal imaging. For example, the emissivity of hair-covered skin may vary and will therefore not provide accurate surface temperature when measured using thermal imaging.

When using thermography to detect health conditions of an animal, the effect of e.g. emissivity implies that reference data captured for an individual animal may not be re-used on other animals having different surface properties. Furthermore, if the surface properties of an animal change due to e.g. hair growth or age, then the predicted statistics will be obsolete.

### SUMMARY

It is an object of the disclosure to alleviate at least some of the drawbacks with the prior art. Thus, it is an object to provide a method for determining a health condition of an animal based on surface temperature, which is not affected by surface properties of the body surface of the animal, or at least less affected than previously known solutions.

According to a first aspect, the disclosure relates to a method for determining a health condition of an animal. The method comprises capturing a thermographic image of at least one portion of the animal and capturing a visible light image of the at least one portion of the animal, wherein the visible light image corresponds to the thermographic image. The method further comprises determining at least one surface property of the at least one portion of the animal based on the visible light image, adjusting the thermographic image to compensate for impact of the determined at least one surface property and determining the health condition of the animal based on the adjusted thermographic image. By using a visible light image to adjust the thermographic image, effects caused by properties of the animal's body surface may be mitigated and a more accurate determination of the health condition is achieved. Furthermore, many thermographic cameras on the market today already comprise a visible light sensor, that is used for other purposed. Hence, the proposed method may be implemented without addition of hardware.

In some embodiments, the at least one surface property is indicative of emissivity and wherein the adjusting comprises compensating the thermographic image to eliminate impact of variations of the emissivity in the surface of the at least one portion of the animal. Thereby, deficiencies in the determination of the health condition that are caused by varying emissivity of the animal's body surface will be mitigated.

In some embodiments, the determining comprises determining the at least one surface property for each of a plurality of image segments in the visible light image and wherein the adjusting comprises adjusting corresponding image segments in the thermographic image to compensate the thermographic image for impact of variations of the at least one surface property. Thereby, all image segments will be adjusted to a common reference scale, which is independent of the surface properties.

In some embodiments, the determining comprises normalising thermographic values of corresponding image segments in the thermographic image to a common reference emissivity, based on the determined emissivity values of the plurality of image segments. Thereby, all image segments will be adjusted to a common reference emissivity, which is independent of the surface properties.

In some embodiments, the image segments are pixels or groups of pixels. Thereby, the adjusting may be done on different levels of granularity depending on the particular use case.

In some embodiments, the at least one surface property comprises at least one of colour, light, texture, wetness, dirt and hairiness. Thereby, these properties will not affect the determination of the health condition.

In some embodiments, the visible light image is an RGB image. Thereby, a standard camera may be used to capture the visible light image.

In some embodiments, the thermographic image and the visible light image are aligned or correlated in space and time. Thereby, an accurate adjustment of the thermographic image is possible.

According to a second aspect, the disclosure relates to a control unit configured to determine a health condition of an animal. The control unit is configured to obtain a thermographic image of at least one portion of the animal and obtain a visible light image of the at least one portion of the animal, wherein the visible light image corresponds to the thermographic image. The control unit is further configured to determine at least one surface property of the at least one portion of the animal based on the visible light image; to adjust the thermographic image to compensate for impact of the determined at least one surface property, and to determine the health condition of the animal based on the adjusted thermographic image.

In some embodiments, the at least one surface property is indicative of emissivity and wherein the control unit is configured to adjust the thermographic image by compensating the thermographic image to eliminate impact of variations of the emissivity in the surface of the at least one portion of the animal.

In some embodiments, the control unit is configured to determine the at least one surface property for each of a plurality of image segments in the visible light image and to adjust the corresponding image segments in the thermographic image to compensate the thermographic image for impact of variations of the at least one surface property.

In some embodiments, the control unit is configured to determine an emissivity value for each of the plurality of image segments of the visible light image and to normalize thermographic values of corresponding image segments in the thermographic image to a common reference emissivity, based on the corresponding determined emissivity values of the plurality of image segments.

In some embodiments, the image segments are pixels or groups of pixels. In some embodiments, the at least one surface property comprises at least one of colour, light, texture, wetness, dirt and hairiness. In some embodiments, the visible light image is an RGB image. In some embodiments, the thermographic image and the visible light image are aligned or correlated in space and time.

According to a third aspect, the disclosure relates to a computer program comprising instructions which, when the program is executed by a computer, cause the computer to carry out the method according to the first aspect.

According to a fourth aspect, the disclosure relates to a computer-readable medium comprising instructions which, when executed by a computer, cause the computer to carry out the method according to the first aspect.

### BRIEF DESCRIPTION OF THE DRAWINGS

FIG. 1 illustrates a vision system, where the proposed technique may be implemented.

FIG. 2 illustrates a visible light image of a portion of an animal.

FIG. 3 is a flowchart of the proposed method for determining a health condition of an animal according to the first aspect.

FIG. 4 illustrates control unit according to the second aspect.

### DETAILED DESCRIPTION

It is previously known to detect an anomaly of an animal by comparing predicted statistics for an individual subject and measurements made upon that subject at any given time. However, temperature measured by a thermal camera is influenced by surface conditions of the body as surface properties may affect the emissivity of the body surface. Hence, in animal body temperature measurement, colour, thickness of hair etc. will influence the measured temperature, as the surface properties may affect the emissivity or reflectivity of the body surface. In multi-coloured dairy cattle the effect of varying emissivity may be even more serious as body parts of different colour, e.g. black and white patterns in Holstein, give significantly different measured temperature results, although the actual body temperature is the same in both black and white parts.

Hence, variation in emissivity or reflectivity may be caused by surface properties such as hair, colour, dirt, roughness etc. Thus, without considering variation in emissivity or reflectivity caused by varying surface properties, a thermographic camera may have low accuracy or not be applicable for animal monitoring, especially for multi-coloured dairy animal, such as Holstein. In addition, thermographic images of a body part of an animal may be influenced by which body part is imaged. Hence, if the body part is not known, this may also affect the results, as the surface temperature typically differ between body parts.

This disclosure proposes a solution where a visible light camera is used to compensate a thermographic image for varying surface properties. The solution includes to use the visible light camera to detect properties of the animal that may influence the thermographic measurements performed on an animal, in order to compensate a thermographic image of the animal to mitigate effects caused by these properties. In some embodiments, the visible light camera may be used to detect colour, hairiness, dirt etc. and other properties that may influence the emissivity and to compensate the thermographic image for varying emissivity. In this way a thermographic image which is less influenced by emissivity is obtained. Such an image is suitable for use when determining a health condition of an animal.

FIG. 1 illustrates a vision system 100 where the proposed method for determining a health condition of an animal may be implemented. The vision system 100 comprises an image sensor arrangement 50 and a control unit 40.

The image sensor arrangement 50 is basically a camera configured to capture thermographic (not shown) and visible light images of a portion of an animal 1. In other words, the image sensor arrangement 50 comprises a visible light image sensor and a thermographic image sensor. The visible light image sensor and a thermographic image sensor may be arranged in the same housing (as in the illustrated example), or they may be physically separated. The visible light image sensor may be configured to capture visible light images with colour information, i.e. colour images. Colour images are typically suitable for detecting body surface properties such as hairiness or colour. In this example, the image sensor arrangement 50 is arranged to capture a visible light image and a thermographic image of a portion 10 being an udder of a cow.

The visible light image sensor and a thermographic image sensor are configured to capture images that are aligned or correlated in space and time. Hence, the visible light image sensor and a thermographic image sensor may be mechanically aligned, or they may be aligned by software, provided that at least one reference point is known in both images. In other words, the relation between one pixel or image segment in the visible light image sensor and a corresponding pixel or image segment in the thermographic image sensor is known. The mapping may be a 1:1 mapping, or any other mapping.

The control unit 40, or simply controller, is a computing device configured to perform the proposed method for determining a health condition of an animal 1. The control unit 40 may either be implemented within, or in connection to, the image sensor arrangement 50. The control unit 40 may also (at least partly) be implemented in a server that is located at a remote location.

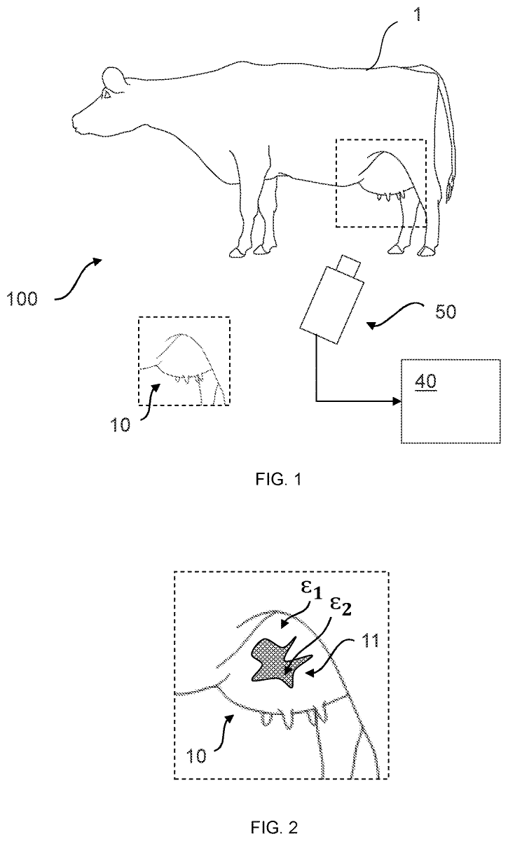

FIG. 2 illustrates an example visible light image captured by a visible light image sensor 50. The visible light image pictures a portion 11 of an animal, or more specifically an udder of a cow. In the example image there is a dark area at the surface 11 of the udder. The dark area typically has a higher emissivity ε₁, than the rest of the udder, which has a lower emissivity ε₂. Hence, in a thermographic image the dark area may appear to have a higher temperature, even if the actual surface temperature is the same at the entire udder. Thus, in this situation, it is useful to calibrate the thermographic image to compensate for emissivity E using the method that will now be proposed.

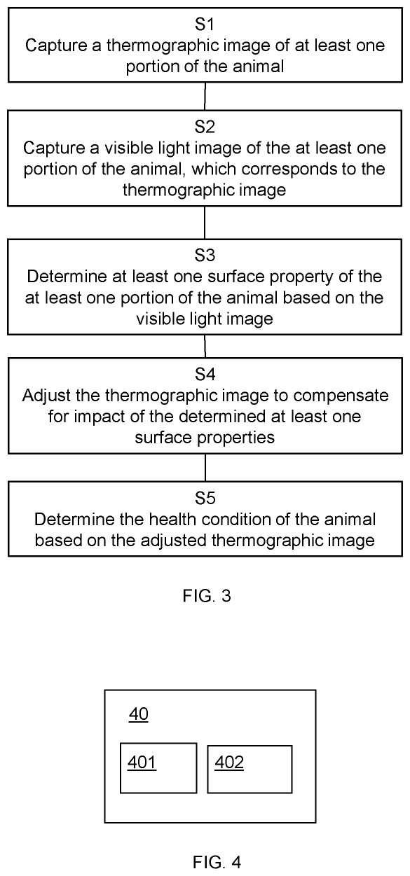

The proposed technique will now be described in further detail with reference to the flow chart of FIG. 3 and the vision system of FIG. 1. FIG. 3 is a flow chart of the proposed method for determining a health condition of an animal. The method of FIG. 3 is e.g. performed by a control unit 40 (FIG. 4) of a vision system 100 (FIG. 1). The method may be implemented as a computer program comprising instructions which, when the program is executed by a computer (e.g. a processor in the control unit 40 (FIG. 4)), cause the computer to carry out the method. According to some embodiments the computer program is stored in a computer-readable medium (e.g. a memory or a compact disc) that comprises instructions which, when executed by a computer, cause the computer to carry out the method.

The method is typically performed when checking an animal for a health condition. A health condition is e.g. a disease or injury. For example, a cow is positioned in front of the image sensor arrangement 50 of FIG. 1 to investigate if it has mastitis. The image sensor arrangement 41 may be handheld or it may be fixed to the surrounding (as in FIG. 1).

The method comprises capturing S1 a thermographic image of at least one portion of the animal 1. For example, the control unit 40 triggers the image sensor arrangement 50 to capture the thermographic image, e.g. by sending a control signal to the image sensor arrangement 50. The visible light image would typically be an ordinary RGB image, but it may also be a monochrome image or other type of image suitable for detecting surface properties of a body surface of an animal that may affect emissivity and/or reflectivity of the animal's body surface. The visible light image may in some embodiments in addition or alternatively be suitable to detect different parts of the animal's body. The portion 11 of the animal 1 is e.g. and udder, a teat, a side, the back, the behind, a muzzle, a nostril, a hair pattern, a patch of skin, a hoof, a mouth, genitalia, a part thereof or a combination thereof, or the like. The animal 1 is e.g. livestock, a cow, a sheep, a pig, a horse, a deer, or any other animal.

The method further comprises capturing S2 a visible light image of the at least one portion 10 of the animal 1. The visible light image corresponds to the thermographic image. That the first image corresponds to the second image implies that if the position (e.g. pixel) of one point of the animal 1 (e.g. corner of a teat) is known in the visible light image, then that position is also present, and can be identified, in the thermographic image. Hence, there is a known relationship between the images. Typically, the images are also captured at the same point in time, or at least very close in time (less than a second in-between). In other words, the first and second images are aligned or correlated in space and time.

The visible light image is used to reveal properties of the body surface of the animal 1 that may influence the thermographic image as for example, the colour of the animal or thickness of its fur may affect the thermographic imaging. In other words, the method comprises determining S3 at least one surface property of the at least one portion 10 of the animal 1, based on the visible light image. Examples of surface properties are colour, light exposure, texture, dirtiness, roughness and hairiness.

The surface properties are for example calculated per pixel or per group of pixel, or for other image segments. In other words, in some embodiments, the determining S3 comprises, determining the at least one surface property for each of a plurality of image segments in the visible light image. The determination is done in different ways for different properties. Some surface properties such as colour or dirt may easily be extracted from the image data of the visible light image. Note that this may be done without using any reference object. However, it may require that the visible light image is captured under controlled lighting conditions. Then, the lightest pixel in the thermographic image may simply be assumed to be white, or some other suitable colour.

Other properties, such as hair may be detected using commonly known techniques for feature detection. The different properties may be combined such that for each image segment (e.g. pixel) an emissivity value εₑₛₜ is estimated. In other words, in some embodiments, the determining S3 comprises, determining an emissivity value for each of the plurality of image segments of the visible light image. For example, a table is used to translate a certain colour and/or hair thickness to a corresponding estimated emissivity, as illustrated in Table 1. Such a look-up table may be created based on reference data.

TABLE 1
Mapping between colour and estimated emissivity

| Colour | Emmisivity (εₑₛₜ) |
|---|---|
| White | 0.84 |
| Yellow | 0.87 |
| Brown | 0.90 |
| Black | 0.95 |

Alternatively, the determining S3 may use a trained model to determine an emissivity value for a certain set of surface properties. Thus, a model may be defined that takes a set of surface properties as input and provides an emissivity value as output. Such a model may be continuously updated when more data is collected.

The thermographic image may then be calibrated to mitigate effects of the determined body surface properties. For example, for image segments having a high estimated emissivity εₑₛₜ the thermographic value is reduced. In other words, the method comprises adjusting S4 the thermographic image to compensate for impact of the determined at least one surface property.

In one example implementation, the at least one surface property is indicative of emissivity. For example, the surface property is indicated by an estimated emissivity value εₑₛₜ, as in table 1. The adjusting S4 then comprises compensating the thermographic image to eliminate impact of variations of the emissivity in the surface 11 of the at least one portion 10 of the animal 1. The adjusting S4 then comprises adjusting corresponding image segments in the thermographic image to compensate the thermographic image for impact of variations of the at least one surface property. For example, the temperature of each pixel is divided by the corresponding emissivity value εₑₛₜ. In this way a measured temperature of a dark image segment will be reduced in relation to a measured temperature of a lighter image segment and thereby influence of emissivity is mitigated.

One way of compensating the thermographic image is to normalize the thermographic values of the thermographic image to a common reference emissivity ε_{ref}. This basically means that the thermographic values of the image segments are rescaled such that they can be compared to each other. For example, the temperature values of all the pixels are recalculated to similar surface properties, e.g. white colour and no hair. Normalisation may be made using the formula (1).

$${T}_{n o r m} = {T}_{meas} * \frac{{ɛ}_{ref}}{{ɛ}_{e s t}} \qquad ( 1 )$$

This means for example that the temperature value of pixels in dark areas is reduced, as those areas emit more efficiently than lighter areas. An example of how pixel temperatures of a thermographic image may be normalised to reference emissivity of 0.95 (corresponding to black colour) is presented in Table 2.

TABLE 2
Normalisation of measured temperatures
based on estimated emissivity

| Measured temp. | Emmisivity | Normalised |
|---|---|---|
| (Tₘₑₐₛ) | (εₘₑₐₛ) | temp. (Tₙₒᵣₘ) |
| 340 | 0.87 | 37.12 |
| 350 | 0.90 | 36.9 |
| 370 | 0.95 | 37 |

In other words, in some embodiments, the adjusting comprises normalising thermographic values of corresponding image segments in the thermographic image to a common reference emissivity ε_{ref}, based on the corresponding determined emissivity values εₘₑₐₛ of the plurality of image segments.

In some embodiments, the at least one surface property is a property indicative of reflectivity e.g. roughness. The adjusting S4 then comprises compensating the thermographic image to eliminate impact of variations of the reflectivity in the surface 11 of the at least one portion 10 of the animal 1.

Furthermore, the visible light image may also be used to compensate for the fact that there is a correlation between the animal's inner temperature and measured surface temperature is affected by on which body part the temperature is measured. As an example, a knee of a cow is typically colder than the udder. Hence, in some embodiments, the at least one surface property is indicative of which body part is imaged. The adjusting S4 then comprises compensating the thermographic image to eliminate impact of which body part is imaged in the thermographic image.

The method comprises determining S5 the health condition of the animal 1 based on the adjusted thermographic image. For example, an anomaly may be detected by comparing the adjusted thermographic with predicted statistics. If the deviation between the predicted and measured results greater than a predetermined threshold, the measurement is regarded as an anomaly.

FIG. 4 illustrates the control unit 40 in more detail. The control unit 40 comprises hardware and software. The hardware is for example various electronic components on a for example a Printed Circuit Board, PCB. The most important of those components is typically a processor 401 e.g. a microprocessor, along with a memory 402 e.g. EPROM or a Flash memory chip. The software (also called firmware) is typically lower-level software code that runs in the microcontroller. The control unit 40 comprises a communication interface, e.g. I/O interface or other communication bus, for communicating with the image sensor arrangement 50. In some embodiments the communication interface is wireless.

The control unit 40, or more specifically a processor 401 of the control unit 40, is configured to cause the control unit 40 to perform all aspects of the method described in FIG. 3. This is typically done by running computer program code stored in the memory 402 in the processor 401 of the control unit 40.

More particularly, the control unit 40 is configured to determine a health condition of an animal 1. The control unit 40 is configured to obtain a thermographic image of at least one portion 10 of the animal 1 and to obtain a visible light image of the at least one portion 10 of the animal 1, wherein the visible light image corresponds to the thermographic image. In some embodiments, the visible light image is an RGB image. In some embodiments, the thermographic image and the visible light image are aligned or correlated in space and time.

Furthermore, the control unit 40 is configured to determine at least one surface property of the at least one portion 10 of the animal 1, based on the visible light image, to adjust the thermographic image to compensate for impact of the determined at least one surface property and to determine the health condition of the animal 1 based on the adjusted thermographic image. In some embodiments, the at least one surface property comprises at least one of colour, light, texture, wetness, dirt and hairiness.

In some embodiments, the at least one surface property is indicative of emissivity and the control unit 40 is configured to adjust the thermographic image by compensating the thermographic image to eliminate impact of variations of the emissivity in the surface 11 of the at least one portion 10 of the animal 1.

In some embodiments, the control unit 40 is configured to determine the at least one surface property for each of a plurality of image segments in the visible light image and to adjust the corresponding image segments in the thermographic image to compensate the thermographic image for impact of variations of the at least one surface property.

In some embodiments, the control unit 40 is configured to determine an emissivity value for each of the plurality of image segments of the visible light image and to normalize thermographic values of corresponding image segments in the thermographic image to a common reference emissivity. In some embodiments, the image segments are pixels or groups of pixels.

The terminology used in the description of the embodiments as illustrated in the accompanying drawings is not intended to be limiting of the described method; control arrangement or computer program. Various changes, substitutions and/or alterations may be made, without departing from disclosure embodiments as defined by the appended claims.

The term “or” as used herein, is to be interpreted as a mathematical OR, i.e., as an inclusive disjunction; not as a mathematical exclusive OR (XOR), unless expressly stated otherwise. In addition, the singular forms “a”, “an” and “the” are to be interpreted as “at least one”, thus also possibly comprising a plurality of entities of the same kind, unless expressly stated otherwise. It will be further understood that the terms “includes”, “comprises”, “including” and/or “comprising”, specifies the presence of stated features, actions, integers, steps, operations, elements, and/or components, but do not preclude the presence or addition of one or more other features, actions, integers, steps, operations, elements, components, and/or groups thereof. A single unit such as e.g. a processor may fulfil the functions of several items recited in the claims.

1. A method for determining a health condition of an animal, the method comprising:
capturing a thermographic image of at least one portion of the animal;
capturing a visible light image of the at least one portion of the animal, the visible light image corresponding to the thermographic image;
determining at least one surface property of the at least one portion of the animal based on the visible light image;
adjusting the thermographic image to compensate for an impact of variations of the determined at least one surface property in a surface of the at least one portion of the animal to obtain an adjusted thermographic image; and
determining the health condition of the animal based on the adjusted thermographic image.

2. The method according to claim 1, wherein at least one surface property is indicative of emissivity, and
wherein the adjusting comprises compensating the thermographic image to eliminate the impact of variations of the emissivity in the surface of the at least one portion of the animal.

3. The method according to claim 1, wherein the determining comprises determining the at least one surface property for each of a plurality of image segments in the visible light image, and
wherein the adjusting comprises adjusting corresponding image segments in the thermographic image to compensate the thermographic image for the impact of variations of the at least one surface property.

4. The method according to claim 3, wherein the determining comprises determining an emissivity value for each of the plurality of image segments of the visible light image, and
wherein the adjusting comprises normalizing thermographic values of corresponding image segments in the thermographic image to a common reference emissivity, based on the corresponding determined emissivity values of the plurality of image segments.

5. The method according to claim 3, wherein the image segments are pixels or groups of pixels.

6. The method according to claim 1, wherein the at least one surface property comprises at least one of color colour, light, texture, wetness, dirt, and hairiness.

7. The method according to claim 1, wherein the visible light image is an RGB image.

8. The method according to claim 1, wherein the thermographic image and the visible light image are aligned or correlated in space and time.

9. A computer program product embodied on a non-transitory computer-readable medium, the computer program product comprising instructions which, when the program is executed by a computer, cause the computer to carry out the method according to claim 1.

10. A non-transitory computer-readable medium comprising instructions which, when executed by a computer, cause the computer to carry out the method according to claim 1.

11. A controller configured to determine a health condition of an animal, the controller comprising:
one or more processors configured to
obtain a thermographic image of at least one portion of the animal,
obtain a visible light image of the at least one portion of the animal, the visible light image corresponding to the thermographic image,
determine at least one surface property of the at least one portion of the animal based on the visible light image,
adjust the thermographic image to compensate for an impact of variations of the determined at least one surface property in a surface of the at least one portion of the animal to obtain an adjusted thermographic image, and
determine the health condition of the animal based on the adjusted thermographic image.

12. The controller according to claim 11, wherein the at least one surface property is indicative of emissivity, and
wherein the controller is configured to adjust the thermographic image by compensating the thermographic image to eliminate impact of variations of the emissivity in a surface of the at least one portion of the animal.

13. The controller according to claim 11, wherein the controller is configured to determine the at least one surface property for each of a plurality of image segments in the visible light image and to adjust the corresponding image segments in the thermographic image to compensate the thermographic image for an impact of variations of the at least one surface property.

14. The controller according to claim 11, wherein the controller is configured to determine an emissivity value for each of the plurality of image segments of the visible light image and to normalize thermographic values of corresponding image segments in the thermographic image to a common reference emissivity, based on the corresponding determined emissivity values of the plurality of image segments.

15. The controller according to claim 11, wherein the image segments are pixels or groups of pixels.

16. The controller according to claim 11, wherein the at least one surface property comprises at least one of color, light, texture, wetness, dirt, and hairiness.

17. The controller according to claim 11, wherein the visible light image is an RGB image.

18. The controller according to claim 11, wherein the thermographic image and the visible light image are aligned or correlated in space and time.

19. The method according to claim 1, wherein the adjusting the thermographic image to compensate for an impact of variations of the determined at least one surface property comprises rescaling thermographic values within the thermographic image such that the thermographic values are able to be compared to one another to obtain the adjusted thermographic image.

20. A method for determining a health condition of an animal, the method comprising:
capturing a thermographic image of at least one portion of the animal;
capturing a visible light image of the at least one portion of the animal, the visible light image corresponding to the thermographic image;
determining at least one surface property of the at least one portion of the animal based on the visible light image, the at least one surface property being a specific body part of the animal that is imaged;
adjusting the thermographic image to compensate for the at least one surface property based on the specific body part that is imaged in relation to other body parts of the animal to obtain an adjusted thermographic image; and
determining the health condition of the animal based on the adjusted thermographic image.
